(19) United States
(12) Patent Application Publication
(10) Pub. No.: US 2004/0243085 A1
(43) Pub. Date: Dec. 2, 2004

(54) Disposable absorbent article having an integral waistband

(75) Inventors: Jerome Steven Veith, Menasha, WI (US); Paul VanGompel, Hortonville, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/449,943

(22) Filed: May 30, 2003

(51) Int. Cl. A61F 13/15; A61F 13/20

(52) U.S. Cl. 604/385.3

(57) ABSTRACT

A disposable absorbent article is disclosed that includes front and back panels each having a first zone and a second zone. The first zone is extensible and retractable and the second zone is non-extensible and non-retractable. The disposable absorbent article also has a waistband integrally formed in the first zone that has extensible regions separated by non-extensible regions. The waistband is constructed such that a greater force is required to extend it than is required to extend the remaining extensible portion of the first zone in both the front and back panels. The disposable absorbent article also includes an absorbent assembly and the front and back panels are secured together by a pair of seams to form a waist opening and a pair of leg openings.

### BACKGROUND OF THE INVENTION

A disposable absorbent article is designed for absorbing human exudate. The disposable absorbent article is similar in appearance, size and shape to a regular cloth underwear except that it is not designed to be laundered and reused two or more times. A disposable absorbent article is intended to be worn by persons, including infants, toddlers, or adults, and is designed for single or temporary use. The disposable absorbent article is meant to be disposed of after being used once. The disposable absorbent article is designed to be pulled up around the user's torso without having to first open the garment in order to place it on a person's body. The stretchability of the material used to construct the disposable absorbent article permits the garment to snugly conform to the anatomy of the user's torso. The disposable absorbent article can be manufactured to be an infant diaper, a child training pant, an adult incontinence garment, a feminine menstrual pant, etc.

Some disposable absorbent articles manufactured today resemble regular cloth underwear in that they have a waist opening and a pair of leg openings. Such disposable absorbent articles can be pulled up around the torso of a user in a similar fashion as regular cloth underwear. It has been found that a disposable absorbent article is generally more discreet when the front and back elastic panels snugly conform to the wearer's anatomy. It has also been found that a more comfortable article is obtained when the waistband has sufficient tension to firmly hold the article about the user's torso even when the article is retaining a large amount of body fluid and/or excrement. An integral waistband is advantageous to prevent noticeable areas of increased thickness or bulges that may be apparent under the user's external clothing.

Now a disposable absorbent article for absorbing human exudate has been invented that incorporates an integral waistband that has sufficient tension to firmly hold the absorbent article about the user's torso, even after the article has been insulted with a large quantity of body fluid and/or excrement.

### SUMMARY OF THE INVENTION

Briefly, this invention relates to a disposable absorbent article that includes front and back panels each having a first zone and a second zone. The first zone is extensible and retractable and the second zone is non-extensible and non-retractable. Each of the front and back panels also has an inner surface, a first end, and first and second side edges, and the second zone is spaced inward from the first and second side edges. The disposable absorbent article also has a waistband integrally formed in the first zone and located adjacent to the first end of both the front and back panels. The waistband has multiple extensible regions each separated by a non-extensible region. Each of the extensible regions has a length that is at least equal to the length of the non-extensible regions. The waistband is constructed such that a greater force is required to extend it than is required to extend the remaining extensible portion of the first zone in both the front and back panels. An absorbent assembly bridges across the front and back panels. The absorbent assembly includes a liquid pervious bodyside liner, a liquid-impervious outer cover, and an absorbent positioned therebetween. The absorbent assembly has a first end and a second end. The absorbent assembly is secured to the inner surfaces of the front and back panels. The first end of the absorbent assembly is secured to the second zone of the front panel and the second end of the absorbent assembly is secured to the second zone of the back panel. The absorbent assembly is capable of being folded to enable the first and second side edges of the front panel to align with the first and second side edges of the back panel. A pair of seams join the front and back panels together at the first and second side edges to form a disposable absorbent article having a waist opening and a pair of leg openings.

### BRIEF DESCRIPTION OF THE DRAWINGS

FIG. 1 is a perspective view of a disposable absorbent article.

FIG. 2 is a top view of the spaced apart front and back panels showing the first and second zones.

FIG. 3 is an end view of the front panel of FIG. 2 taken along line 3--3.

FIG. 4 is an end view of the back panel of FIG. 2 taken along line 4--4.

FIG. 5 is a side view of the front panel shown in FIG. 2.

FIG. 6 is a side view of the back panel shown in FIG. 2.

FIG. 7 is a top view of the front panel shown in FIG. 2 after the inner layer has been removed exposing the multiple elastic strands and a portion of the integral waistband.

FIG. 8 is an enlarged view of the area circled in FIG. 7 depicting a portion of two of the elastic strands.

FIG. 9 is a plane view of a disposable absorbent article showing an absorbent assembly secured to the inner surfaces of the front and back panels and showing a partial cut away view of the construction of the absorbent assembly.

FIG. 10 is a side view of the disposable absorbent article shown in FIG. 9.

### DETAILED DESCRIPTION

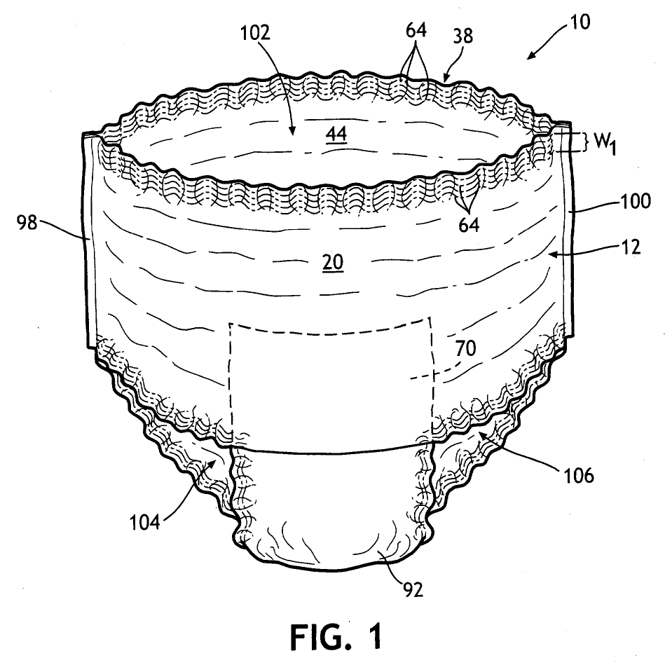

Referring to FIG. 1, a disposable absorbent article 10 is depicted. The disposable absorbent article 10 is intended to be worn by persons, including infants, toddlers, or adults, and is designed for a single or temporary use. The disposable absorbent article 10 is meant to be disposed of after being used once instead of being laundered or dry cleaned for re-use. The disposable absorbent article 10 is designed to be pulled up around the user's torso without having to first open the article 10 in order to place it on a person's body. In FIG. 1, the disposable absorbent article 10 is shown as it would appear just prior to being pulled up around a user's torso.

Referring to FIGS. 2-6, the disposable absorbent article 10 has a longitudinal central axis X--X, a transverse central axis Y--Y, and a vertical central axis Z--Z. The disposable absorbent article 10 includes a front panel 12 having a first zone 14 and a second zone 16. The first zone 14 is extensible and retractable in at least one direction. Desirably, the first zone 14 is extensible and retractable in two or more directions. When the first zone 14 is extensible and retractable in only one direction, that direction should be approximately parallel to the transverse central axis Y--Y of the disposable absorbent article 10. The second zone 16 is non-extensible and non-retractable. The second zone 16 can be of any geometrical configuration but a rectangular or square configuration works well. The second zone 16 has a surface area that is smaller than the surface area of the first zone 14. Desirably, the second zone 16 has a surface area that is less than about 25% of the surface area of the first zone 14. More desirably, the second zone 16 has a surface area that is less than about 20% of the surface area of the first zone 14. Most desirably, the second zone 16 has a surface area that is less than about 1.5% of the surface area of the first zone 14.

Still referring to FIGS. 2, 3 and 5, the front panel 12 also includes an inner surface 18 and an outer surface 20. The inner surface 18 is in direct contact with the user's skin and is sometimes referred to as the bodyside surface. The outer surface 20 is situated opposite to the inner surface 18 and is spaced away from the skin of the user. The outer surface 20 is sometimes referred to as the garment facing surface since it can be in direct contact with the inner surfaces of the user's outer clothing.

Referring to FIG. 2, the front panel 12 further includes a first end 22, a second end 24, a first side edge 26 and a second side edge 28. The overall size and shape of the front panel 12 can vary to suit the size and anatomy of the actual user. For example, the front panel 12 of an infant diaper will be smaller than the front panel 12 of an adult incontinence garment. The first and second zones, 14 and 16 respectively, can be integrally formed from a single material or they can be formed from separate and distinct materials. The second zone 16 is spaced inward from the first and second side edges 26 and 28. The second zone 16 is also shown having an edge that is coterminuous with the second end 24 of the front panel 12. The second zone 16 is located adjacent to or in an abutting relationship to the first zone 14. However, the second zone 16 should not overlay or be covered by any portion of the first zone 14. Desirably, the second zone 16 is surrounded on three sides by the first zone 14, as is shown in FIG. 2.

The front panel 12 can be formed from a material that is extensible and retractable in at least one direction. By “extensible” it is meant a material that is capable of being stretched, extended or elongated when a force, such as pulling, is applied to it. By “retractable” it is meant a material that is capable of at least partially recovering, retracting or becoming shorter once the force used to extend the material has been removed. Because of hysteresis, the material may not be able to fully recover or return to its original pre-stretched length. Some materials, including but not limited to, a cotton T-shirt material or blends of cotton and non-elastic synthetic fibers can be extended but are not considered retractable for the purpose of this invention unless some elasticizing material, agent or treatment is added.

The front panel 12 can be formed from an elastomeric material. An elastomeric material can be a single elastic sheet or layer, an elastic film, an elastic net-like material, a plurality of elastic strands arranged to form an elastic layer, an elastic laminate, etc. When a plurality of elastic strands is utilized, they can be positioned on at least one layer of woven or non-woven material. When an elastic laminate is used, it can consist of two or more layers bonded together by heat, pressure, heat and pressure, adhesives, ultrasonics, or a combination of any of the above. Other means of securing one or more layers together to form a laminate structure are known to those skilled in the art. In a laminate, only one of the layers needs to be an elastomeric layer.

Referring now to FIGS. 3 and 5, the front panel 12 is shown being formed as a three-layer laminate structure. The front panel 12 includes a first layer 30, a second or middle layer 32, and a third layer 34. The first and third layers, 30 and 34 respectively, are the outer layers and can be formed from the same material or from different materials. The first and third layers, 30 and 34 respectively, can be constructed from natural or synthetic fibers and can be a woven or non-woven material. The second or middle layer 32 has the elastic properties and is sandwiched between the first and third layers, 30 and 34 respectively. It should be noted that one or both of the outer layers 30 or 34 can be made from an elastic material, if desired.

The extensible and retractable properties of the first zone 14 of the front panel 12 can be in one direction but desirably are in two or more directions. More desirably, the extensible and retractable properties of the first zone 14 are in a direction approximately parallel to the transverse central axis Y--Y. The transverse direction extends laterally across the torso of the user of the disposable absorbent article 10 and extends from one hip bone to the other hip bone. Even more desirably, the extensible and retractable properties of the first zone 14 are in at least two directions, one direction being approximately parallel to the longitudinal central axis X--X and the other direction being approximately parallel to the transverse central axis Y--Y. Most desirably, the extensible and retractable properties of the first zone 14 are in multiple directions, or stated another way, in three or more directions extending over an arc of 360 degrees. The ability of the first zone 14 to extend and retract will provide the requisite force needed during use of the disposable absorbent article 10 to ensure that it snugly conforms to the anatomy of the wearer's torso.

Referring to FIGS. 3 and 5, the front panel 12 can be formed from two outer layers 30 and 34 with a plurality of elastic strands 36 sandwiched therebetween. The elastic strands 36 can be formed from LYCRA®. LYCRA® is a registered trademark of E. I. Du Pont De Nemours & Co., having an office at 1007 Market Street, Wilmington, Del. 19898. The elastic strands 36 can be aligned approximately parallel to one another or be angled or skewed relative to one another. The elastic strands 36 can also be uniformly or randomly spaced apart from one another. The elastic strands 36 can vary in shape, size, configuration, and/or length. The diameter and/or cross-sectional configuration of the elastic strands 36, the decitex (weight in grams per 10,000 meters of a strand) of the elastic strands 36, and the tension imparted into the elastic strands 36 can all be varied to suit one's particular product needs. The elastic strands 36 can have a round, semi-circular, square, rectangular, oval or some other geometrical configuration. The elastic strands 36 can overlap, intersect or crisscross at least one other elastic strand 36. The various ways of positioning, orienting, and adhering the elastic strands 36 to the two outer layers 30 and 34 are well known to those skilled in the art.

The front panel 12 can also be constructed from various materials. One suitable material is a stretch bonded laminate (SBL) where the elastic core or middle layer 32 is elongated before the two outer nonwoven layers 30 and 34 are attached. Exemplary SBL materials are described in U.S. Pat. No. 4,720,415 which is hereby incorporated by reference and made a part hereof. Another suitable material for the front panel 12 is a necked bonded laminate (NBL). The NBL material is also a three-layer laminate but the elastic core or middle layer 32 is not pre-stretched prior to being attached to the two outer nonwoven layers 30 and 34. Instead, the outer layers 30 and 34 are necked stretched before the elastic core or middle layer 32 is attached to them. Exemplary NBL materials are described in U.S. Pat. No. 5,336,545 which is hereby incorporated by reference and made a part hereof. Other examples of such elastomeric materials that can be used for the front panel 12 include a continuous filament stretch bonded laminate (CFSBL), a vertical filament laminate (VFL), a necked stretch bonded laminate (NSBL) and a necked thermal laminate (NTL). Combinations of the above materials can also be used. Exemplary CFSBL materials are described in U.S. Pat. No. 5,385,775 which is hereby incorporated by reference and made a part hereof.

Furthermore, the front panel 12 can be constructed from an elastic film that is capable of being stretched in at least one direction. Desirably, the front panel 12 can be stretched in both the machine direction (approximately parallel to the longitudinal central axis X--X) and the cross-direction (approximately parallel to the transverse central axis Y--Y). The front panel 12 can also be formed from an elastic nonwoven that has a machine direction stretch and/or a cross-direction stretch.

It should be noted that the front panel 12 can also be constructed from a material that is substantially air permeable, if desired. Alternatively, the front panel 12 can be constructed from a material that is substantially air impermeable.

The front panel 12 can be formed from a single integral elastomeric material that exhibits both extensible and retractable characteristics. The material can then be treated, fused, deadened, chopped, conditioned or somehow altered so as to form the second zone 16. Hammering the material between two hardened surfaces can also cause the material to lose its extensible and retractable properties in a given area. The material can also be treated with heat, pressure, heat and pressure, ultrasonics, chemicals, by mechanical means, or a combination of the above to remove its ability to elongate and retract. In this fashion, the second zone 16 can be formed. Alternatively, the second zone 16 can be a separated material that does not possess any extensible and retractable properties. In this case, the second zone 16 is secured to the first zone 14 in order to construct the front panel 12.

Referring now to FIGS. 2, 4 and 6, the disposable absorbent article 10 also includes a back panel 38 which is spaced apart, discontinuous and distinct from the front panel 12. The back panel 38 has a first zone 40 and a second zone 42. The first zone 40 is extensible and retractable in at least one direction. Desirably, the first zone 40 is extensible and retractable in two or more directions. When the first zone 40 is extensible and retractable in only one direction, that direction should be approximately parallel to the transverse central axis Y--Y of the disposable absorbent article 10. The second zone 42 is non-extensible and non-retractable. The second zone 42 can be of any geometrical configuration but a rectangular or square configuration works well. The second zone 42 has a surface area that is smaller than the surface area of the first zone 40. Desirably, the second zone 42 has a surface area that is less than about 30% of the surface area of the first zone 40. More desirably, the second zone 42 has a surface area that is less than about 25% of the surface area of the first zone 40. Most desirably, the second zone 42 has a surface area that is less than about 20% of the surface area of the first zone 40.

It should be noted that the second zone 42 of the back panel 38 has a larger surface area than the second zone 16 of the front panel 12. One reason for this is that the front panel 12 usually has a smaller overall surface area than the back panel 38. The back panel 38 is typically larger in size for it has to cover the buttocks of the user.

Still referring to FIGS. 2, 4 and 6, the back panel 38 also includes an inner surface 44 and an outer surface 46. The inner surface 44 is in direct contact with the user's skin and is sometimes referred to as the bodyside surface. The outer surface 46 is situated opposite to the inner surface 44 and is spaced away from the skin of the user. The outer surface 46 is sometimes referred to as the garment facing surface since it can be in direct contact with the inner surfaces of the user's outer clothing.

The back panel 38 further includes a first end 48, a second end 50, a first side edge 52 and a second side edge 54. The overall size and shape of the back panel 38 can vary to suit the size and anatomy of the actual user. For example, the back panel 38 for an infant diaper will be smaller than the back panel 38 for an adult incontinence garment. The first and second zones, 40 and 42 respectively, of the back panel 38 can be integrally formed from a single material or they can be formed from separate and distinct material. The second zone 42 is spaced inward from the first and second side edges 52 and 54. The second zone 42 is also shown having an edge that is coterminuous with the second end 50 of the back panel 38. The second zone 42 is located adjacent to or in an abutting relationship to the first zone 40. However, the second zone 42 should not overlay or be covered by any portion of the first zone 40. Desirably, the second zone 42 is surrounded on three sides by the first zone 40, as is shown in FIG. 2.

The back panel 38 can be formed from the same material or from a different material as the front panel 12. The back panel 38 is extensible and retractable in at least one direction. The definitions for “extensible and retractable” are as defined above with reference to the front panel 12. The back panel 38 can be formed from an elastomeric material. An elastomeric material can be a single elastic sheet or layer, an elastic film, an elastic net-like material, a plurality of elastic strands arranged to form an elastic layer, an elastic laminate, etc. When a plurality of elastic strands is utilized, they can be positioned on at least one layer of woven or non-woven material. When an elastic laminate is used, it can consist of two or more layers bonded together by heat, pressure, heat and pressure, adhesives, ultrasonics, or a combination of any of the above. Other means of securing one or more layers together to form a laminate structure are known to those skilled in the art. In a laminate, only one of the layers needs to be an elastomeric layer.

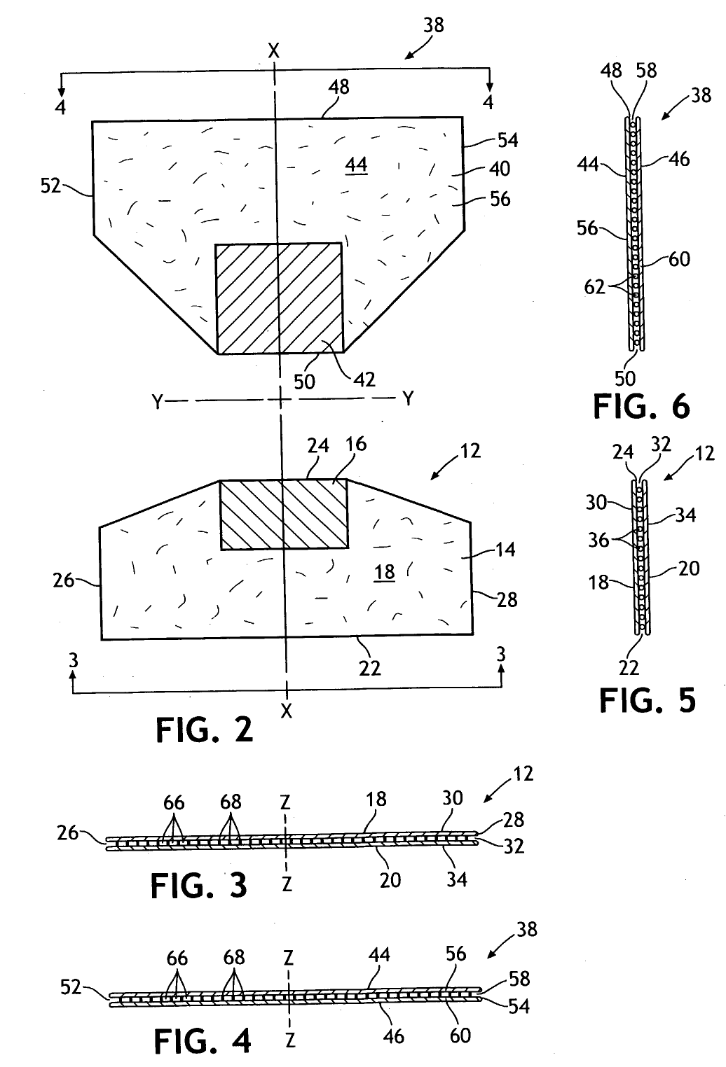

Referring again to FIGS. 4 and 6, the back panel 38 is shown being formed as a three-layer laminate structure. The back panel 38 includes a first layer 56, a second or middle layer 58, and a third layer 60. The first and third layers, 56 and 60 respectively, are the outer layers and can be formed from the same material or from different materials. The first and third layers, 56 and 60 respectively, can be constructed from natural or synthetic fibers and can be a woven or non-woven material. The second or middle layer 58 has the elastic properties and is sandwiched between the first and third layers, 56 and 60 respectively. It should be noted that one or both of the outer layers 56 or 60 can be made from an elastic material, if desired.

The extensible and retractable properties of the first zone 40 of the back panel 38 can be in one direction but desirably are in two or more directions. More desirably, the extensible and retractable properties of the first zone 40 are in a direction approximately parallel to the transverse central axis Y--Y. The transverse direction extends laterally across the torso of the user of the disposable absorbent article 10 and extends from one hip bone to the other hip bone. Even more desirably, the extensible and retractable properties of the first zone 40 are in at least two directions, one direction being approximately parallel to the longitudinal central axis X--X and the other direction being approximately parallel to the transverse central axis Y--Y. Most desirably, the extensible and retractable properties of the first zone 40 are in multiple directions, or stated another way, in three or more directions extending over an arc of 360 degrees. The ability of the first zone 40 to extend and retract will provide the requisite force needed during use of the disposable absorbent article 10 to ensure that it snugly conforms to the anatomy of the wearer's torso.

Referring to FIG. 6, the back panel 38 can be formed from two outer layers 56 and 60 with a plurality of elastic strands 62 sandwiched therebetween. The elastic strands 62 can be formed from LYCRA®. LYCRA® is a registered trademark of E. I. Du Pont De Nemours & Co., having an office at 1007 Market Street, Wilmington, Del. 19898. The elastic strands 62 can be aligned approximately parallel to one another or be angled or skewed relative to one another. The elastic strands 62 can also be uniformly or randomly spaced apart from one another. The elastic strands 62 can vary in shape, size, configuration, and/or length. The diameter and/or cross-sectional configuration of the elastic strands 62, the decitex (weight in grams per 10,000 meters of a strand) of the elastic strands 62, and the tension imparted into the elastic strands 62 can all be varied to suit one's particular product needs. The elastic strands 62 can have a round, semi-circular, square, rectangular, oval or some other geometrical configuration. The elastic strands 62 can overlap, intersect or crisscross at least one other elastic strand 62. The various ways of positioning, orienting, and adhering the elastic strands 62 to the two outer layers 56 and 60 are well known to those skilled in the art.

The back panel 38 can also be constructed from various materials. One suitable material is a stretch bonded laminate (SBL) where the elastic core or middle layer 58 is elongated before the two outer nonwoven layers 56 and 60 are attached. Another suitable material for the back panel 38 is a necked bonded laminate (NBL). The NBL material is also a three-layer laminate but the elastic core or middle layer 58 is not pre-stretched prior to being attached to the two outer nonwoven layers 56 and 60. Instead, the outer layers 56 and 60 are necked stretched before the elastic core or middle layer 58 is attached to them. Other examples of such elastomeric materials that can be used for the back panel 38 include a continuous filament stretch bonded laminate (CFSBL), a vertical filament laminate (VFL), a necked stretch bonded laminate (NSBL) or a necked thermal laminate (NTL). Combinations of the above materials can also be used.

Furthermore, the back panel 38 can be constructed from an elastic film that is capable of being stretched in at least one direction and, desirably, in both the machine direction (approximately parallel to the longitudinal central axis X-X) and the cross-direction (approximately parallel to the transverse central axis Y-Y). The back panel 38 can also be formed from an elastic nonwoven that has a machine direction stretch and/or a cross-direction stretch.

It should be noted that the back panel 38 can also be constructed from a material that is substantially air permeable, if desired. Alternatively, the back panel 38 can be constructed from a material that is substantially air impermeable.

The back panel 38 can be formed from a single integral elastomeric material that exhibits both extensible and retractable characteristics. The material can then be treated, fused, deadened, chopped, conditioned or somehow altered so as to form the second zone 42. Hammering the material between two hardened surfaces can also cause the material to lose its extensible and retractable properties in a given area. The material can also be treated with heat, pressure, heat and pressure, ultrasonics, chemicals, by mechanical means, or a combination of the above to remove its ability to elongate and/or retract. In this fashion, the second zone 42 can be formed. Alternatively, the second zone 42 can be a separated material that does not possess any extensible and retractable properties. In this case, the second zone 42 is secured to the first zone 40 in order to construct the back panel 38.

Referring again to FIG. 2, one can see that the first zone 14 of the front panel 12 encloses three sides of the second zone 16. Likewise, the first zone 40 of the back panel 38 encloses three sides of the second zone 42. This construction ensures that the second zones 16 and 42 are situated adjacent to an end, 24 or 50 respectively, of the front and back panels, 12 and 38 respectively.

Referring now to FIGS. 1-4, 7 and 8, the disposable absorbent article 10 also includes a waistband 64 integrally formed in the first zones 14 and 40 of the front and back panels, 12 and 38 respectively. By “integrally formed” it is meant that the waistband 64 is formed from the same material used to form the first zones 14 and 40 of the front and back panels, 12 and 38 respectively. Desirably, the waistband 64 has a thickness that is approximately equal to the thickness of the remaining portion of the first zones 14 and 40 of the front and back panels, 12 and 38 respectively. The waistband 64 is not formed by folding the material over or upon itself, or by attaching or securing a separate strip or piece of material onto the front and back panels, 12 and 38 respectively. The waistband 64 does not bulge or extend outward in the z-direction from the front and back panels, 12 and 38 respectively. The waistband 64 is located adjacent to the first ends 22 and 48 of the front and back panels, 12 and 38 respectively. The waistband 64 has a width w₁ measured perpendicular to the first ends 22 and 48 of the front and back panels, 12 and 38 respectively. The width w₁ of the waistband 64 has a dimension that is less than about 2 inches (about 5 cm). Desirably, the width w₁ of the waistband 64 has a dimension that is less than about 1.5 inches (about 3.8 cm). More desirably, the width w₁ of the waistband 64 has a dimension that ranges from about 0.25 inches (about 0.6 cm) to about 1.5 inches (about 3.8 cm).

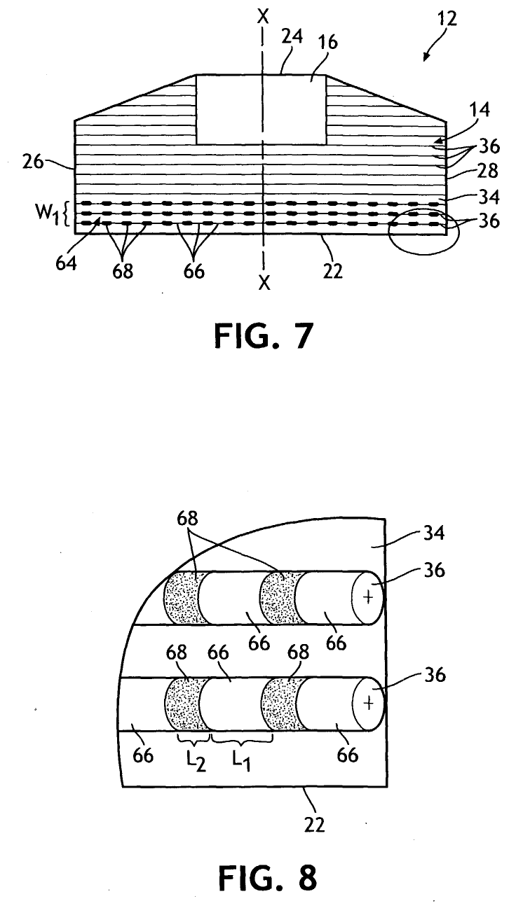

Referring to FIGS. 7-10, the waistband 64 includes multiple extensible regions 66 each separated by a non-extensible region 68. The extensible regions 66 can be a portion of one or more elastic strands 36 or 62 or an area of an elastic sheet. Each of the extensible regions 66 has a length L₁ and each of the non-extensible regions 68 has a length L₂. The length L₁ of each of the extensible regions 66 is at least equal to the length L₂ of each of the non-extensible regions 68. Desirably, the length L₁ of each of the extensible regions 66 is longer than the length L₂ of each of the non-extensible regions 68. More desirably, the length L₁ of each of the extensible regions 66 is at least 2 times longer than the length L₂ of each of the non-extensible regions 68. Still more desirably, the length L₁ of each of the extensible regions 66 is at least 3 times longer than the length L₂ of each of the non-extensible regions 68. Most desirably, the length L₁ of each of the extensible regions 66 is at least 4 times longer than the length L₂ of each of the non-extensible regions 68.

Still referring to FIGS. 7-10, the non-extensible regions 68 of the waistband 64 have been deactivated. By “deactivated” it is meant that the elastic has been rendered inactive or ineffective. The elastic strands 36 and 62 can be deactivated by using heat, pressure, heat and pressure, ultrasonic energy, a combination of any of the aforementioned, etc. Other ways of deactivating the elastic that may be known to those skilled in the art can be used.

Referring to FIGS. 7 and 8, the waistband 64 includes multiple elastic strands 36. Desirably, from about 2 to about 15 elastic strands 36 can be present across the width w₁ of the waistband 64. More desirably, from about 3 to about 10 elastic strands 62 are present across the width w₁ of the waistband 64. Still more desirably, from about 4 to about 8 elastic strands 36 are present across the width w₁ of the waistband 64. Most desirably, at least 5 elastic strands 36 are present across the width w₁ of the waistband 64.

Each of the elastic strands 36 and 62 located in the front and back panels, 12 and 38 respectively, is capable of being extended or stretched in at least one direction. Desirably, when only one direction of extension or stretch is present, that direction will be aligned approximately parallel to the transverse axis Y--Y. The extensible regions 66 of each elastic strand 36 and 62 is free to stretch or elongate as a tension force is applied to the front and back panels, 12 and 38 respectively. However, the non-extensible regions 68 are not free to stretch or elongate because the elastic has been deactivated. Therefore, the non-extensible regions 68 represent areas or zones wherein the elastic has been locked up, deadened or destroyed such that these regions do not have the ability to stretch or elongate. Because the non-extensible regions 68 are located adjacent to the extensile regions 66, their positions will necessitate a greater force than is needed to expand or enlarge the waistband 64. Therefore, the force needed to extend the waistband 64 from its initial size will be greater than the force needed to extend the remaining extensible portions of the first zones 14 and 40 of the front and back panels, 12 and 38 respectively. Desirably, the force needed to extend the waistband 64 from its initial size will be at least 1.5 times greater than the force needed to extend the remaining extensible portions of the first zones 14 and 40 of the front and back panels, 12 and 38 respectively. More desirably, the force needed to extend the waistband 64 from its initial size will be at least 2 times greater than the force needed to extend the remaining extensible portions of the first zones 14 and 40 of the front and back panels, 12 and 38 respectively.

The requirement that a greater tension force is needed to extend or expand the size of the waistband 64 from its initial size will assure that the waistband 64 will assert a sufficient force on the torso of the user of the disposable absorbent article 10 to hold it in position even after the disposable absorbent article 10 has absorbed and is retaining a larger quantity of body fluid or excrement. The secure fit provided by the waistband 64 will provide the wearer with peace of mind in knowing that the disposable absorbent article 10 will not droop or slide down on his or her torso.

Referring again to FIGS. 1, 9 and 10, the disposable absorbent article 10 also includes an absorbent assembly 70. The absorbent assembly 70 includes a liquid pervious bodyside liner 72, a liquid-impervious outer cover 74, and an absorbent 76 positioned therebetween. A surge layer 78 can be optionally used, which is located between the bodyside liner 72 and the absorbent 76. The surge layer 78 can function to rapidly acquire and temporarily retain body fluid, such as urine, before it can be absorbed into the absorbent 76. Desirably, the surge layer 78 is also capable of wicking the body fluid lengthwise and/or widthwise across its surface, as well as directing the body fluid downward in a z-direction (approximately parallel to the vertical axis Z-Z) toward the absorbent 76.

The absorbent assembly 70 has a first end 80, a second end 82, a first side edge 84 and a second side edge 86. The absorbent assembly 70 is secured to the inner surface 18 of the front panel 12 approximate the first end 80 by an attachment 88 and is secured to the inner surface 44 of the back panel 38 approximate the second end 82 by an attachment 90. The absorbent assembly 70 is secured to the front and back panels, 12 and 38 respectively, after each panel has been stretched a predetermined amount. By attaching the absorbent assembly 70 to the inner surfaces 18 and 44 of the front and back panels, 12 and 38 respectively, the absorbent assembly 70 is capable of moving downward away from the user's torso while the front and back panels, 12 and 38 respectively, maintain their snug position against the user's torso. This unique ability for the absorbent assembly 70 to freely move outward and downward away from the user's torso without undue restriction from the front and back panels, 12 and 38 respectively, produces a useful undergarment. The absorbent assembly 70 is capable of taking in and retaining additional body fluid as it moves out away from the user's body.

The attachments 88 and 90 can be by various means and can include permanent attachments as well as removable or releasable attachments. Desirably, the attachments 88 and 90 are permanent attachments where they are not designed to be removed without destroying the bond. The attachments 88 and 90 can be formed by using glue, adhesive, ultrasonic bonds, heat bonds, pressure bonds, heat and pressure bonds, a combination of any of the aforementioned, etc. The attachments 88 and 90 can also include a mechanical fastener, such as by sewing with thread, using buttons and button holes, using snaps, by employing hook and loop fasteners, etc. A hook and loop fastener is generally considered a releasable attachment. One type of hook and loop fastener is VELCRO® wherein a hook material is releasably engaged into a loop material. VELCRO® is a registered trademark of Velcro USA, Inc. having an office at 406 Brown Avenue, Manchester, N.H. 03103.

The attachments 88 and 90 can be formed along a continuous line or over a surface area having a predetermined length and width. Alternatively, the attachments 88 and 90 can consist of intermittent point bonds that are spaced apart from one another. For example, the intermittent point bonds can be formed by using a hot or cold melt adhesive or by forming ultrasonic bonds. Various bond formations can be used which are known to those skilled in the art. Desirably, the attachments 88 and 90 are formed using intermittent bonds.

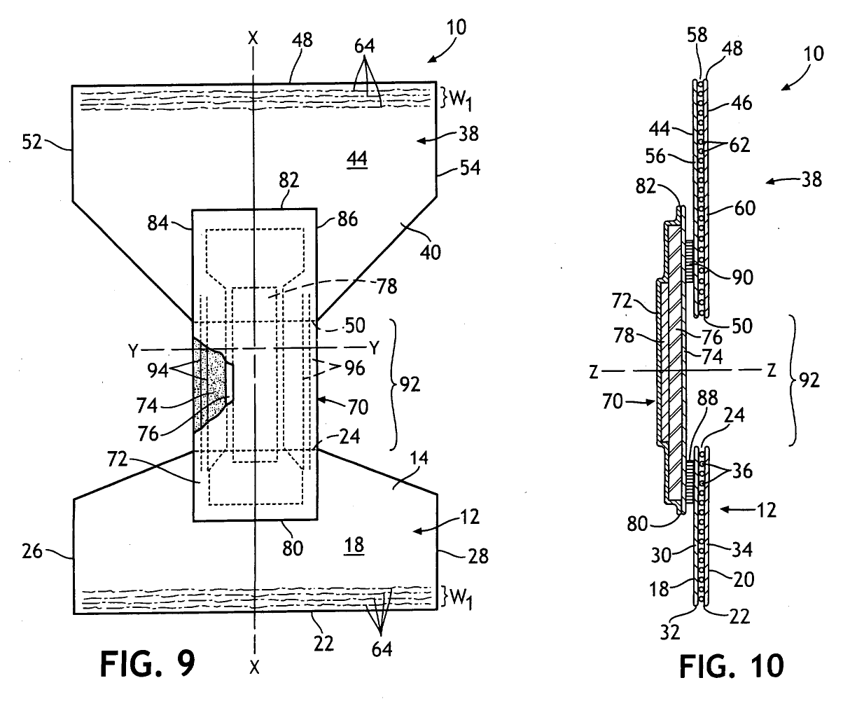

Referring again to FIGS. 2, 9 and 10, one will notice that the absorbent assembly 70 is positioned to overlay the second zones 16 and 42 of the front and back panels, 12 and 38 respectively. Each of the second zones 16 and 42 can have the same width dimension or a width of a different dimension. Desirably, each of the second zones 16 and 42 has a width of the same dimension. The width of the absorbent assembly 70 can be greater than, equal to or less than the width of either of the second zones 16 and 42. However, the absorbent assembly 70 is secured only to the second zones 16 and 42 so as not to extend and retract with the movement of the first zones 14 and 40 of the front and back panels, 12 and 38 respectively. This method of attachment will assure that the absorbent assembly 70 remains relatively stationary while the front and back panels, 12 and 38 respectively, are capable of extending and retracting in correspondence to movement of the user's torso. In FIGS. 9 and 10, one will also notice that the absorbent assembly 70 is positioned such that a greater portion of it is located over the back panel 38 than over the front panel 12. This placement of the absorbent assembly 70 over a greater portion of the back panel 38 provides a better functioning disposable absorbent article 10, especially one that is designed to absorb both body fluid from the penis or vagina, as well as solid and semi-solid excrement from the anus. In addition, the front panel 12 is usually smaller in size than the back panel 38. This size difference also influences the placement of the absorbent assembly 70 over a greater portion of the back panel 38. The result is that the first end 80 of the absorbent assembly 70 is secured to the front panel 12 at a location that is closer to the second end 24 of the front panel 12 then it is to the first end 22. Likewise, the second end 82 of the absorbent assembly 70 is secured to the back panel 38 at a location that is closer to the second end 50 of the back panel 38 then to the first end 48.

Still referring to FIGS. 9 and 10, one will notice that when the absorbent assembly 70 is secured to the front and back panels, 12 and 38 respectively, a crotch region 92 is formed. The crotch region 92 separates the front panel 12 from the back panel 38 and is designed to cover the perineum area of the wearer. The crotch region 92 can cover a distance of a few inches in an infant diaper to several inches in an adult incontinence garment. For example, a crotch region 92 in an infant diaper may range from about 2 inches (about 5 centimeters(cm)) to about 10 inches (about 25 cm), while in an adult incontinence garment; the crotch region 92 may range from about 6 inches (about 15 cm) to about 20 inches (about 51 cm).

The absorbent assembly 70 can be stretchable or non-stretchable in relation to the front and back panels, 12 and 38 respectively. Desirably, the absorbent assembly 70 is non-stretchable in relation to the front and back panels, 12 and 38 respectively. By having the absorbent assembly 70 be non-stretchable in relation to the front and back panels, 12 and 38 respectively, it is meant that the absorbent assembly 70 will not stretch appreciably in the longitudinal or transverse directions. The reason for this is that the front and back panels, 12 and 38 respectively, are elastically stretchable and can expand and contract to snugly conform to the user's anatomy, especially to the wearer's torso. The absorbent assembly 70 is designed not to expand and contract as the front and back panels, 12 and 38 respectively, stretch or retract since the absorbent assembly 70 is bonded to the non-extensible and non-retractable second zones 16 and 42. This feature allows the absorbent assembly 70 to remain positioned over the user's perineum. As the absorbent assembly 70 receives body fluid and/or excrement discharged by the wearer, it will be displaced outward and downward, away from the user's torso. The ability of the absorbent assembly 70 to move outward away from the user's torso as additional body fluid is absorbed and retained is a direct result of the fact that it is not restricted from such movement in the crotch region 92 by the front or back panels, 12 and 38 respectively. The attachments 88 and 90 assure that the absorbent assembly 70 covers the perineum but is capable of moving outward away from the torso as additional body fluid is received and retained.

Still referring to FIGS. 9 and 10, the disposable absorbent article 10 also has at least one elastic member 94 positioned adjacent to and aligned approximately parallel the first side edge 84 and at least one elastic member 96 positioned adjacent to and aligned approximately parallel the second side edge 86 of the absorbent assembly 70. Each of the elastic members 94 and 96 is situated between the bodyside liner 72 and the outer cover 74. The elastic members 94 and 96 provide a gasket to hold the first and second side edges, 84 and 86 respectively, of the absorbent assembly 70 against the user's body. Each of the elastic members 94 and 96 can be in the form of an elastic strand, ribbon or strip. Desirably, from two to six elastic members 94 and 96 will be positioned adjacent to each of the first and second side edges, 84 and 86 respectively. In FIG. 9, there are two elastic members 94 positioned adjacent to the first side edge 84 and two elastic members 96 positioned adjacent to the second side edge 86. Desirably, the elastic members 94 and 96 have a round cross-sectional configuration although various other geometrical configurations can be utilized. The elastic members 94 and 96 extend approximately parallel to the longitudinal central axis X--X and extend completely through the crotch region 92. Desirably, the ends of the elastic members 94 and 96 will be located within the front and back panels, 12 and 38 respectively.

Referring again to FIGS. 1, 9 and 10, the absorbent assembly 70 is capable of being folded transversely, approximate the transverse central axis Y--Y, to enable the first and second side edges 26 and 28 of the front panel 12 to align with the first and second side edges 52 and 54 of the back panel 38, respectively. A pair of seams 98 and 100 is then formed to join the front panel 12 to the back panel 38. The seam 98 secures the first side edge 26 of the front panel 12 to the second side edge 52 of the back panel 38 while the seam 100 secures the second side edge 28 of the front panel 12 to the second side edge 54 of the back panel 38. One will notice that FIG. 9 is an open view of the interior of the disposable absorbent article 10 and the front panel 12 is folded along the transverse central axis Y--Y such that the front panel 12 lies over the top of the back panel 38. After folding and forming the pair of seams 98 and 100, the disposable absorbent article 10 shown in FIG. 1 is obtained. The disposable absorbent article 10 has a waist opening 102 and a pair of leg openings 104 and 106. Since the front and back panels, 12 and 38 respectively, are formed from a stretchable elastic material, the waist opening 102 and the pair of leg openings 104 and 106 can expand or contract in size to accommodate the anatomy of the user.

While the invention has been described in conjunction with a specific embodiment, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable absorbent article comprising:
a) a front panel having a first zone and a second zone, said first zone being extensible and retractable and said second zone being non-extensible and non-retractable, said front panel also having an inner surface, a first end, and first and second side edges, and said second zone being spaced inward from said first and second side edges;
b) a back panel having a first zone and a second zone, said first zone being extensible and retractable and said second zone being non-extensible and non-retractable, said back panel also having an inner surface, a first end, and first and second side edges; and said second zone of said back panel being spaced inward from said first and second side edges of said back panel;
c) a waistband integrally formed in said first zone and located adjacent to said first end of both said front and back panels, said waistband including multiple extensible regions each separated by a non-extensible region, each of said extensible regions having a length that is at least equal to the length of said non-extensible regions, and said waistband requiring a greater force to extend than is required to extend the remaining extensible portion of said first zone in both said front and back panels;
d) an absorbent assembly including a liquid pervious bodyside liner, a liquid-impervious outer cover, and an absorbent positioned therebetween, said absorbent assembly having a first end and a second end, said absorbent assembly being secured to said inner surfaces of said front and back panels, said first end of said absorbent assembly being secured to said second zone of said front panel and said second end of said absorbent assembly being secured to said second zone of said back panel, and said absorbent assembly capable of being folded to enable said first and second side edges of said front panel to align with said first and second side edges of said back panel; and
e) a pair of seams joining said front and back panels together at said first and second side edges to form a disposable absorbent article having a waist opening and a pair of leg openings.

2. The disposable absorbent article of claim 1 wherein each of said extensible regions of said waistband has a length that is longer than the length of said non-extensible regions of said waistband.

3. The disposable absorbent article of claim 2 wherein each of said extensible regions of said waistband has a length that is at least 2 times longer than the length of said non-extensible regions of said waistband.

4. The disposable absorbent article of claim 3 wherein each of said extensible regions of said waistband has a length that is at least 3 times longer than the length of said non-extensible regions of said waistband.

5. The disposable absorbent article of claim 1 wherein the force needed to extend said waistband is at least 1.5 times greater than the force needed to extend the remaining extensible portion of said first zone in both said front and back panels.

6. The disposable absorbent article of claim 1 wherein said force needed to extend said waistband is at least 2 times greater than the force needed to extend the remaining extensible portion of said first zone in both said front and back panels.

7. The disposable absorbent article of claim 1 wherein said non-extensible regions of said waistband contain elastic that has been deactivated.

8. The disposable absorbent article of claim 7 wherein said elastic has been deactivated by heat.

9. The disposable absorbent article of claim 7 wherein said elastic has been deactivated by ultrasonic energy.

10. A disposable absorbent article comprising:
a) a front panel having a first zone and a second zone, said first zone being extensible and retractable and said second zone being non-extensible and non-retractable, said front panel also having an inner surface, a first end, and first and second side edges, and said second zone being spaced inward from said first and second side edges;
b) a back panel having a first zone and a second zone, said first zone being extensible and retractable and said second zone being non-extensible and non-retractable, said back panel also having an inner surface, a first end, and first and second side edges, and said second zone of said back panel being spaced inward from said first and second side edges of said back panel;
c) a waistband integrally formed in said first zone and located adjacent to said first end of both said front and back panels, said waistband including multiple elastic strands each having extensible regions separated by non-extensible regions, each of said extensible regions having a length that is longer than the length of said non-extensible regions, and said waistband requiring a greater force to extend than is required to extend the remaining extensible portion of said first zone in both said front and back panels;
d) an absorbent assembly including a liquid pervious bodyside liner, a liquid-impervious outer cover, and an absorbent positioned therebetween, said absorbent assembly having a first end and a second end, said absorbent assembly being secured to said inner surfaces of said front and back panels, said first end of said absorbent assembly being secured to said second zone of said front panel and said second end of said absorbent assembly being secured to said second zone of said back panel, and said absorbent assembly capable of being folded to enable said first and second side edges of said front panel to align with said first and second side edges of said back panel; and
e) a pair of seams joining said front and back panels together at said first and second side edges to form a disposable absorbent article having a waist opening and a pair of leg openings.

11. The disposable absorbent article of claim 10 wherein said non-extensible regions of said waistband are deactivated by heat.

12. The disposable absorbent article of claim 10 wherein said non-extensible regions of said waistband are deactivated by pressure.

13. The disposable absorbent article of claim 10 wherein said non-extensible regions of said waistband are deactivated by ultrasonic energy.

14. The disposable absorbent article of claim 10 wherein said multiple elastic strands of said waistband are extensible in one direction.

15. The disposable absorbent article of claim 10 wherein said waistband contains at least 5 elastic strands.

16. A disposable absorbent article comprising:
a) a front panel having a first zone and a second zone, said first zone being extensible and retractable and said second zone being non-extensible and non-retractable, said front panel also having an inner surface, a first end, and first and second side edges, and said second zone being spaced inward from said first and second side edges;
b) a back panel having a first zone and a second zone, said first zone being extensible and retractable and said second zone being non-extensible and non-retractable, said back panel also having an inner surface, a first end, and first and second side edges, and said second zone of said back panel being spaced inward from said first and second side edges of said back panel;
c) a waistband integrally formed in said first zone and located adjacent to said first end of both said front and back panels, said waistband including multiple extensible regions each separated by a non-extensible region, each of said extensible regions having a length that is at least 3 times the length of said non-extensible regions, and said waistband requiring a greater force to extend than is required to extend the remaining extensible portion of said first zone in both said front and back panels;
d) an absorbent assembly including a liquid pervious bodyside liner, a liquid-impervious outer cover, and an absorbent positioned therebetween, said absorbent assembly having a first end and a second end, said absorbent assembly being secured to said inner surfaces of said front and back panels, said first end of said absorbent assembly being secured to said second zone of said front panel and said second end of said absorbent assembly being secured to said second zone of said back panel, and said absorbent assembly capable of being folded to enable said first and second side edges of said front panel to align with said first and second side edges of said back panel; and
e) a pair of seams joining said front and back panels together at said first and second side edges to form a disposable absorbent article having a waist opening and a pair of leg openings.

17. The disposable absorbent article of claim 16 wherein said waistband has a width measured perpendicular to said first end that is less than about 2 inches.

18. The disposable absorbent article of claim 17 wherein said waistband has a width measured perpendicular to said first end that is less than about 1.5 inches.

19. The disposable absorbent article of claim 16 wherein said force needed to extend said waistband is at least 1.5 times greater than the force needed to extend the remaining extensible portion of said first zone in both said front and back panels.

20. The disposable absorbent article of claim 16 wherein each of said extensible regions of said waistband has a length that is at least 4 times longer than the length of said non-extensible regions of said waistband.

21. The disposable absorbent article of claim 16 wherein said elastic has been deactivated by pressure.
